(19) United States
(12) Patent Application Publication
(10) Pub. No.: US 2017/0298039 A1
(43) Pub. Date: Oct. 19, 2017

(54) TRIPHASIC SYSTEM FOR DIRECT CONVERSION OF SUGARS TO FURANDICARBOXYLIC ACID

(71) Applicant: AGENCY FOR SCIENCE, TECHNOLOGY AND RESEARCH, Singapore (SG)

(72) Inventors: Guangshun YI, Singapore (SG); Yugen ZHANG, Singapore (SG)

(21) Appl. No.: 15/516,018

(22) PCT Filed: Sep. 29, 2015
(86) PCT No.: PCT/SG2015/050351

§ 371 Date: Mar. 30, 2017

(30) Foreign Application Priority Data
Sep. 30, 2014 (SG) 10201406200X

(51) Int. Cl. C07D 307/68; B01J 19/24

(52) U.S. Cl. CPC C07D 307/68; B01J 19/245

(57) ABSTRACT

There is provided a one-pot process for the conversion of sugars to furancarboxylic acids, such as 2,5-furancarboxylic acid (FDCA), in a triphasic system (e.g. water or tetraethylammonium bromide (TEAB)—methyl isobutyl ketone (MIBK)—water). In this reaction setup, sugars are first converted to 5-hydroxymethylfurfural (HMF) in a first phase. Then HMF is then extracted into a second phase and transferred to a third phase of water. In the third phase HMF is converted to the furancarboxylic acid. The overall acid yields obtainable are between about 78% and 50% for conversion from fructose and glucose, respectively. The invention further relates to an apparatus for the triphasic reaction. The apparatus comprises two chambers which allow for the chemically separated reaction of the sugars and the intermediate of the sugars to form the final product in one process. The process according to the invention may be useful for industrial fabrication.

### TECHNICAL FIELD

The present invention generally relates to a one-pot method of producing furandicarboxylic acid from carbohydrate. The furan dicarboxylic acid is obtained in high yields using a triphasic reaction system. The inventions further relates to the apparatus used for the process that allows for the direct production of furandicarboxylic acid from sugars.

### BACKGROUND ART

At the current rate of consumption, the world crude oil reserves can only last for several decades. Therefore, there is an urgent need to develop renewable and sustainable alternatives for fuels and chemicals. The use of renewable biomass, e.g., lignocellulose, presents itself as a good alternative for the production of biofuels and bio-chemicals.

In an application, the use of biomass-based 2,5-furandicarboxylic acid (FDCA) to replace terephthalic acid for the production of polyamides, poly esters, and polyurethanes has received significant attention. In another application, a furan-based polymer poly(ethylene-2,5-furandicarboxylate) (PEF) has been prepared from renewable bio-sources, and it has demonstrated comparable thermal stability to petroleum-based polyethylene terephalate (PET), a polymer commonly made into consumer goods for numerous applications. In view of the above applications, as well as its broad potential as a versatile platform chemical, furandicarboxylic acid is listed as one of the top 12 value-added chemicals from biomass by the United States of America's Department of Energy.

Biomass-derived FDCA is usually produced by a two-step process from sugars or cellulose. However, the second step is very sensitive to the purity of the feedstock. Acidic residual or other impurities from the first step, such as humins, may deactivate the catalyst in the second step, which is usually conducted in a basic environment. As a result, prior to the second step reaction, separation and purification of the intermediate produced from the first step are required. This multiple-step process including thorough separation inevitably leads to high cost, and makes the price of FDCA less competitive than terephthalic acid.

Therefore, a direct conversion of carbohydrates to the final product FDCA would be highly desirable. However, it is a great challenge to directly convert sugars to furandicarboxylic acid, since the conditions for the two-step reactions are conflicting. There is a known method of using a membrane to separate the reactions in a one-pot conversion of fructose to FDCA. However, this method requires several days in reaction time and provides the product only in low yields. In another attempt, cobalt acetylacetonate encapsulated in silica was used to directly convert fructose to FDCA. However, such reaction can only be performed under harsh conditions of high temperature and pressure. Both processes further used fructose as the starting material, while glucose is more favourable due to its abundance and lower price.

There is therefore still a need to develop a more efficient process for direct conversion of low-cost, renewable feedstocks, such as biomass derivatives, to furandicarboxylic acid by a single straightforward fabrication process which may be conducted in a large commercial scale.

Accordingly, there is a need for a process to provide furandicarboxylic acid in high yields, in reasonable reaction times, in the absence of separation steps and in the absence of harsh reaction conditions.

### SUMMARY OF INVENTION

In a first aspect, there is provided a one-pot method of producing furandicarboxylic acid from carbohydrate, the method comprising: a) reacting the carbohydrate via a dehydration reaction to produce an intermediate in a first solvent phase; b) contacting the first solvent phase with a second solvent phase at a first contact area; c) extracting the intermediate to the second solvent phase; d) contacting the second solvent phase directly with a third solvent phase at a different contact area; and e) oxidizing the intermediate to produce the furandicarboxylic acid in the third solvent phase.

Advantageously, this direct tri-phase reaction allows for the production of furandicarboxylic acids in high yields in a simple manner without the need for separation. Reaction times are in the range of several hours and therefore much shorter than in known processes with reaction times of many days.

In one embodiment, the one-pot method is used to convert cellulose, fructose and glucose to furandicarboxylic acid. In another embodiment, fructose and glucose are converted in the method to furandicarboxylic acid. Advantageously, high yields of 78% and 50% respectively can be achieved in this case utilizing the disclosed reaction design.

In one embodiment, 5-hydroxymethylfurfural is the intermediate that is transported between the first solvent phase and the third solvent phase. Advantageously, this intermediate shows a suitable profile to diffuse well from the first solvent phase through the second solvent phase to the third solvent phase.

In another embodiment, an organic solvent selected from C₄₋₆ alkyl alcohol, C₃₋₈ alkyl ketone and mixtures thereof is used as the second solvent phase. This solvent allows for fast diffusion of the intermediate combined with a reduced ability to dissolve furandicarboxylic acid from the third phase therein.

In one embodiment, the oxidation step e) is carried out in the presence of oxygen and a catalytic system. Advantageously, such oxidation can be performed chemically isolated without affecting the reaction in step a).

In a second aspect, there is provided an apparatus for use in converting carbohydrate into furandicarboxylic acid in a one-pot process, the apparatus comprising:

a first chamber fluidly connected to a second chamber by a conduit, wherein the first chamber comprises a dividing means to at least partially separate the first chamber into a first subzone and a second subzone, wherein the first subzone defines a first reaction zone for producing an intermediate from the carbohydrate and the second chamber defines a second reaction zone for producing furandicarboxylic acid from the intermediate, wherein the conduit, the dividing means and the second subzone are configured to at least partially chemically isolate the first and second reaction zones.

Advantageously, the apparatus allows for miming a tri-phasic reaction in an improved design which may not require further structural separating means.

In a third aspect, there is provided a use of the apparatus for converting carbohydrates into furandicarboxylic acid in the one-pot process as disclosed herein.

Definitions

The following words and terms used herein shall have the meaning indicated:

As used herein, the term “chemically isolated” in connection with the method(s) or apparatus refers to the fact that chemical reactions in the separated areas or zones are substantially not influencing each other. There is no substantial effect of chemical reactions in one area or zone on the chemical reactions of the other area or zone except for the transport of reactants from one area or zone to the other.

As used herein, the term “carbohydrate” refers to a saccharide, including sugars, starch, and cellulose. The saccharides can be selected from monosaccharides, disaccharides, oligosaccharides, and polysaccharides.

As used herein, the term “solvent phase” refers to a part of a multiphase system wherein the solvent or a mixture of solvents is substantially uniform and is substantially immiscible with other solvent phases that it is in contact with. The adjacent solvent phases of the multiphase system are separated by a phase boundary which is related to the substantial immiscibility of the solvents.

As used herein, the term “contact area” refers to a phase boundary between the solvent phases.

As used herein, the term “dehydration reaction” refers to a chemical reaction that involves the loss of a water molecule from the reacting molecule.

The term “C₃-C₈ alkyl ketone” refers to a ketone of the general formula alkyl-C(O)-alkyl wherein the alkyl as a group, may be a straight or branched aliphatic hydrocarbon group. The two alkyl groups of the ketone may also contain any number of carbon atoms in the total number range of 3 to 8. Straight and branched alkyl substituents may be selected from the group consisting of methyl, ethyl, propyl, butyl, pentyl, hexyl, heptyl, octyl, nonyl, decyl and any isomers thereof. The alkyl may be selected from the group consisting of methyl, n-ethyl, n-propyl, 2-propyl, n-butyl, sec-butyl, iso-butyl, tert-butyl, n-pentyl, 3-methyl-1-butyl, 2-methyl-1-butyl, 2,2,-dimethyl-1-propyl, 3-pentyl, 2-pentyl, 3-methyl-2-butyl and 2-methyl-2-butyl.

The term “C₄-C₆ alkyl alcohol” refers to an alcohol of the general formula alkyl-OH, wherein the alkyl as a group may be a straight or branched aliphatic hydrocarbon group. The alkyl group of the alcohol may contain any number of carbon atoms in the range of 4 to 6. Straight and branched alkyl substituents may be selected from the group consisting of butyl, pentyl, hexyl, heptyl, octyl, nonyl, decyl and any isomers thereof. The alkyl may be selected from the group consisting of n-butyl, sec-butyl, iso-butyl, tert-butyl, n-pentyl, 3-methyl-1-butyl, 2-methyl-1-butyl, 2,2,-dimethyl-1-propyl, 3-pentyl, 2-pentyl, 3-methyl-2-butyl and 2-methyl-2-butyl.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of the one-pot method will now be disclosed.

There is provided a one-pot method of producing furandicarboxylic acid from carbohydrate, the method comprising:

a) reacting the carbohydrate via a dehydration reaction to produce an intermediate in a first solvent phase; b) contacting the first solvent phase with a second solvent phase at a first contact area; c) extracting the intermediate to the second solvent phase; d) contacting the second solvent phase directly with a third solvent phase at a different contact area; and e) oxidizing the intermediate to produce the furandicarboxylic acid in the third solvent phase.

In step a), the carbohydrate may be one capable of being dehydrated to the intermediate. The carbohydrate may preferably be selected from cellulose, fructose, glucose, any other sugar, or isomers thereof, such as D-glucose or D-fructose. Fructose and glucose may be preferred. Advantageously, the carbohydrate can be obtained from renewable sources such as biomass.

The dehydration reaction may be carried out in the presence of an acid catalyst. The dehydration reaction may be supported by strong acids or strong acid providers, such as ion exchange resins with strongly acidic groups. The reaction may therefore be carried out in the presence of a catalytic system comprising an acidic ion exchange resin. A polystyrene-based ion exchange resin with strongly acidic sulfonic group, such as for instance Amberlyst® 15 (Sigma Aldrich), may be used.

The dehydration reaction may be carried out in the presence of a dehydration agent. In some embodiments, a dehydration agent is used which is able to selectively dehydrate fructose or glucose to 5-hydroxymethylfurfural in a solvent. Mineral acids, Lewis acids and/or salts thereof are suitable. Chromium salts, such as CrCl₃ and CrCl₂ in combination with a quaternary ammonium salt, such as tetraethylammonium bromide (TEAB), can be used. A catalytic system comprising an acidic ion exchange resin and CrCl₃ is most preferred.

The dehydration takes place in the first solvent phase which comprises a solvent that may be selected to be substantially immiscible with the solvent of the second phase. The first solvent phase may be selected to be compatible with the carbohydrate and intermediate. The first solvent phase may be selected such that the carbohydrate and intermediate may be at least partially soluble therein. The first solvent phase may be an aqueous phase. The sole or co-solvent of this phase may be water. In some embodiments, a quaternary ammonium salt, such as tetraethylammonium bromide (TEAB), may alternatively be the sole solvent of the first solvent phase or as a main component in the aqueous solution. Accordingly, the quaternary ammonium salt may be capable of acting as a solvent and as a dehydration agent. The reaction conditions of the dehydration reaction may therefore advantageously be milder, for example, the dehydration reaction may be carried out at a relatively lower reaction temperature as compared to prior art methods while retaining high product yield, when a quaternary ammonium salt is used. In some embodiments, an inorganic salt, such as NaCl, may be added to the aqueous solution of the first solvent phase. In some embodiments, ionic liquids, such as imidazolium salts, can also be used as the sole solvent of the first solvent phase or as a main component in the aqueous solution or as one of the components in the aqueous solution. In some embodiments, the first solvent phase may be an aqueous phase comprising any one or a combination of the above-mentioned salts. In one embodiment, TEAB is used as the solvent of the first solvent phase as it is more economical as compared with ionic liquids and relatively lower reaction temperatures are required. In another embodiment, water is used as the solvent of the first solvent phase.

The dehydration in step a) may be performed at elevated temperatures to dissolve or melt the components of the first solvent phase. The reaction temperature in step a) may be about 90° C. to about 140° C., or about 110° C. to about 130° C., or about 105° C. to about 125° C. The reaction temperature in step a) may be chosen to be at the higher end of the above temperature range, e.g. about 120° C., to increase the rate of reaction. The reaction temperature in step a) may be about 90° C. to about 100° C., with about 95° C. being most preferred. Advantageously, in this embodiment, the temperature of the reaction of step a) may be identical to the preferred or optimized reaction temperature of oxidation step e). Further advantageously, the entire method as disclosed herein may be conducted at the same temperature and the reaction temperature of the entire method as disclosed herein may not require adjustment. In other embodiments, only step a) may be carried out at a temperature of about 110° C. to about 130° C. for 20 to 40 min. The temperature may then be lowered to about 90° C. to about 100° C. so that the temperature of the whole system or both reactions may be adjusted to the same temperature.

The reaction time for step a) may be between about 20 min and 3 hours, preferably about 20 min and 40 min. Step a) may be completed before starting the phase transfer of the product (step b). Step b) may be started before step a) is completed.

The intermediate produced in step a) may comprise one or more carbonyl groups. The carbonyl group may be a ketone or an aldehyde. The dehydration reaction may convert a hydroxyl group of the carbohydrate into a carbonyl group. Where the carbohydrate is fructose or glucose, the intermediate may be hydroxymethylfurfural. The hydroxymethylfurfural may be 5-hydroxymethylfurfural (HMF).

In step b), the first solvent phase is contacted with the second solvent phase via a contact area, termed a “first contact area”. The second solvent phase may be added on top of the first layer of the first solvent phase. The second solvent phase may contact a side of the first solvent phase. The first solvent phase may be added on top or contact the top of the second solvent phase. The first and second solvent phases may be separated by a phase separation in the contact area. The disclosed method may exclude the use of separate or external structural separating means, such as a membrane, at the interface between the first and second solvent phases. As such, the first solvent phase may directly contact the second solvent phase at the first contact area.

In step c), the intermediate produced in the dehydration reaction is extracted into the second solvent phase upon contact of the first and second solvent phases. The solvent of the second solvent phase can be chosen to have a good solubility for the intermediate and allow its fast diffusion therein. The second solvent phase may be selected to allow diffusion of the intermediate through the second solvent phase to the third solvent phase. The second solvent phase may be substantially immiscible with the first solvent phase and the third solvent phase. Preferably the solvent of the second solvent is a substantially water-immiscible organic solvent or a mixture of such solvents. The solvent of the second solvent phase may show a phase separation with the solution of the first solvent phase and the solution of the third solvent phase. It may be selected to at least partially chemically isolate the dehydration step and the oxidation step. The isolation may be achieved by allowing the intermediate to be transported from the first solvent phase to the third solvent phase without influencing the dehydration reaction and the oxidation reaction. The solvent of the second solvent phase may be selected to reduce or prevent furandicarboxylic acid from dissolving therein. The solvent of the second solvent phase may be selected to reduce or prevent the carbohydrate from dissolving therein. Advantageously, the second solvent phase may act as a separating means to segregate the dehydration reaction and the oxidation reaction.

Preferably the solvent of the second solvent phase is poorly miscible with water and shows a phase separation with water. It may be a polar organic solvent with good solubility for the intermediate. It may be a strongly polar molecule, such as ketone or hydrophilic alcohol, but yet is capable of forming a phase separation with the aqueous solution of the first solvent phase. The ketone or hydrophilic alcohol may be for instance an alkyl alcohol or an alkyl ketone. The alkyl group may comprise chains that aid in forming a phase separation with the first solvent phase. For instance, the solvent of the second solvent phase is a C_{4 6} alkyl alcohol, C_{3 8} alkyl ketone or mixtures thereof. Methyl isobutyl ketone (MIBK) and ethyl methyl ketone may be especially mentioned.

The time taken for step c) may include the time taken to extract the intermediate to the second solvent phase and the time taken to diffuse the intermediate through the second solvent phase to the third solvent phase. The time taken for step c) may include the time taken for mass transfer of the intermediate from the first solvent phase to the third solvent phase. The time taken for step c) may be between 5 hours and 55 hours. The time taken for step c) may be controlled by optimizing the selection of the solvent phases and/or the solubility of the components in the respective solvent phases.

The first and second solvent phases may be selected such that the distribution ratio of the intermediate in the solvent phases is of a value suitable to permit the extraction of the intermediate to the second solvent phase. In general, the higher the distribution ratio of the intermediate in the second solvent phase as compared to the first solvent phase, the faster will be the extraction of the intermediate to the second solvent phase. The distribution ratio is defined as the amount of the intermediate in the first solvent phase to the amount of the intermediate in the second solvent phase.

When HMF is the intermediate, the solvent of the second solvent phase is preferably chosen to achieve a distribution ratio of 5-hydroxymethylfurfural in the first solvent phase to the second solvent phase of more than about 0.1. That is, the amount of HMF in the first solvent phase to the amount of HMF in the second solvent phase is about 0.1:1 or more, e.g. about 0.2:1, or about 0.5:1, or about 1:1, or about 2:1, or about 2.7:1, or about 3:1, or about 4:1, or about 5:1. A distribution ratio of about 1.0 to about 5 or about 1.5 to about 3.5 may be preferred.

In the embodiment of a two-phase method, the carbohydrate may be dehydrated to the intermediate in an aqueous layer with acid catalyst. Once formed, the intermediate may be in situ extracted to the top organic layer. In a two-phase method, the intermediate is converted to furandicarboxylic acid in a separate process.

In step d), the second solvent phase is directly contacted with a third solvent phase at a different contact area from the first contact area. This step d) can be concurrently performed with step b) by contacting the second solvent phase at the same time with the other phases at the different contact areas. For example, the second solvent phase may be configured such that it contacts the top layers of the first and third solvent phases. The second solvent phase may contact the bottom layers of the first and third solvent phases. The second solvent phase may contact the sides of the first and third solvent phases. The second solvent phase may contact the other phases non-concurrently or at different times. The second and third solvent phases may be separated by a phase separation in the contact area. The disclosed method may exclude the use of separate or external structural separating means, such as a membrane, at the interface between the second and third solvent phases. While the sequence of the steps is not critical, the following sequence may be advantageous:

First, starting or completing the reaction of step a), then contacting the first solvent phase and third solvent phase with the second solvent phase and finally starting the oxidation reaction of step e).

In step e), the intermediate is oxidized to the final product by generally known methods. The reaction of step e) is performed in a solvent phase that is substantially immiscible with the solvent of the second solvent phase. The third solvent phase may be selected to be compatible with furandicarboxylic acid, or may be capable of at least partially dissolving furandicarboxylic acid therein. As solvent for step e), water may be used. The third solvent phase may be an aqueous solution. A base may be added to the solution. This base may be a carbonate, such as sodium carbonate.

The final product is a furandicarboxylic acid, preferably 2,5-furandicarboxylic acid (FDCA). The solvent of the third solvent phase may be capable of dissolving furandicarboxylic acid produced in the oxidation reaction.

The oxidation in step e) may be carried out in the presence of oxygen and a catalytic system. Oxygen may be bubbled into the third solvent phase. The catalyst may be dissolved or suspended in the third solvent phase. The catalytic system used for this oxidation may be a metal catalyst or a supported metal catalyst. The catalytic system used for this oxidation may be a supported catalytic system comprising gold/hydrotalcite (Au/HT), gold-palladium/hydrotalcite (AU₈Pd₂/HT) or platinum/carbon (Pt/C). For complete oxidation, the catalytic oxidation may be carried out at a temperature of about 30 to 120° C., or about 80 to 110° C. Preferably the reaction temperature is about 90 to 110° C., most preferably it is about 95° C. where optimal conversion to furandicarboxylic acid may be achieved. The reaction time for step e) may be between about 5 and 9 hours, preferably about 7 to 9 hours.

The oxidation of the intermediate in the third solvent phase may be a two-step reaction and may comprise a further step of converting the first intermediate as disclosed above to a second intermediate. The second intermediate may thereafter be converted to furandicarboxylic acid in the third solvent phase. Where the carbohydrate is a C₆ sugar, the second intermediate may be 5-hydroxymethyl-2-furancarboxylic acid (HFCA) produced from HMF and is further oxidized to FDCA. FDCA is the preferred final reaction product.

An example of the reaction pathway of the disclosed method is as follows. Where the carbohydrate is a C₆ sugar, the C₆ sugar is dehydrated to produce 5-hydroxymethylfurfural (HMF). HMF is then oxidized to FDCA with stoichiometric oxidants and metal catalysts or enzymes. The oxidation of HMF may result in HFCA, which is converted to FDCA. The oxidation of HMF to HFCA may be a fast reaction. The reaction scheme of the conversion of HMF to FDCA via HFCA in an aqueous phase is shown in FIG. 1.

Exemplary, non-limiting embodiments of the apparatus will now be disclosed.

The apparatus may be a triphasic reactor, wherein carbohydrates such as sugars can be converted to furandicarboxylic acid such as FDCA in one-pot or one single reactor or one single apparatus. In an example, the apparatus may have three phases (phases I, II, and III), as illustrated in FIG. 2. FIG. 2 shows that sugars are first acid dehydrated to 5-hydroxymethylfurfural (HMF) in phase I. HMF is then extracted, purified and transferred to phase III via a bridge (organic phase II). Finally, HMF is oxidized to FDCA in a base in phase III.

Different types of reactors that can satisfy a triphasic system are shown in FIG. 3. In FIG. 3, phase I comprises the sugar feedstock in TEAB or water for the conversion of the sugar feedstock to the intermediate HMF. Phase II comprises the intermediate HMF in MIBK for extraction and transportation of HMF. Phase III comprises the product FDCA in water for the conversion HMF to FDCA.

FIG. 3A shows an apparatus comprising one chamber comprising a dividing means to partially separate the chamber into a first subzone and a second subzone. In setup A, phase II is placed on top of phases I and III and above the dividing means. However, setup A may not be robust enough to completely separate phase I and phase III when the oxidation reaction in phase III comprises bubbling oxygen and stirring, and thereby result in low efficiency. To overcome this problem, a H-type reactor is provided and shown in FIG. 3B. Setup B comprises a first chamber fluidly connected to a second chamber by a conduit located in the middle of the chambers. Phase II extends through the conduit, while phases I and III are in the respective chambers. However, setup B still may not prevent the different phases from leaking into the other. Furthermore, the efficiency of the HMF mass transfer in phase II may be low in this H-shape setup. FIG. 3C shows another triphasic reactor that may not be able to completely separate phase I and phase III.

Therefore, the present disclosure provides an apparatus for use in converting carbohydrate into furandicarboxylic acid in a one-pot process, the apparatus comprising:

a first chamber fluidly connected to a second chamber by a conduit, wherein the first chamber comprises a dividing means to at least partially separate the first chamber into a first subzone and a second subzone, wherein the first subzone defines a first reaction zone for producing an intermediate from the carbohydrate and the second chamber defines a second reaction zone for producing furandicarboxylic acid from the intermediate, wherein the conduit, the dividing means and the second subzone are configured to at least partially chemically isolate the first and second reaction zones.

FIG. 3D illustrates an apparatus in accordance with an embodiment of the present disclosure. The first subzone in the first chamber comprises phase I. Phase II is placed on top of phases I and III and above the dividing means of the first chamber. The second chamber comprises phase III. Advantageously, the conduit, the dividing means and the second subzone of the first chamber in the embodiment of the present disclosure substantially completely separates phase I and phase III and substantially prevents the different phases from leaking into the other. Therefore, mixing of reactants of the different reactions may advantageously be prevented.

The apparatus may be used in the method as disclosed herein.

The dividing means may be located at any part of the first chamber so long as it can cooperate with the conduit to baffle the fluid passage. The dividing means may extend from the base of the first chamber. The dividing means may have a height suitable to provide an appropriate volume for the first reaction zone. The dividing means may be of a height suitable to allow the second solvent phase to contact the first solvent phase in the first reaction zone and the third solvent phase in the second reaction zone. The dividing means may have a height of about 10% to about 50% of the height of the first chamber. The dividing means physically separates the solution of the first solvent phase from the solution of the third solvent phase. The second solvent phase can be filled on top of the first and third solvent phase in the first chamber.

The dividing means may be made of any suitable material, such as glass. The dividing means may be made of the same material as the apparatus.

The dividing means may be of any shape so long as the dividing means can effectively physically separate the solution of the first solvent phase from the solution of the third solvent phase.

The conduit fluidly connects the first and second chambers. The conduit may be positioned between the first and second chambers at a location suitable to improve the chemical isolation of the first and second reaction zones. The conduit may act together with the dividing means to provide a winding fluid pathway, similar to the action of baffles, to improve the chemical isolation of the first and second reaction zones. The conduit may fluidly connect the first and second chambers at the base. Advantageously, the dividing means that extends from the base of the first chamber cooperates with the conduit at the base of the chambers to provide a meandering passage of fluid flow. In other embodiments, the conduit may be positioned between other locations of the first and second chambers.

The first and second chambers may be cylindrical in shape or rectangular in shape, or any other shapes suitable to contain the solvent phases.

The first and second chambers may be of dimensions suitable to contain an appropriate volume of solvent phases for reaction. The first and second chambers may be of the same dimensions.

In an example, the first and second chambers are cylindrical, each having an internal outer diameter of 23 mm and a height of 75 mm. In another example, the first and second chambers are cylindrical, each having an internal outer diameter of 35 mm and a height of 80 mm. In both examples, the dividing means may be a glass plate separator with a height of 20 mm.

Both chambers may have an opening at the top to fill in the three solvent phases. The conduit may be filled with the solvent of the third solvent phase.

FIG. 5a shows a schematic example of such two chamber set-up with a conduit at the base and a partially separated first chamber. FIG. 5b shows a photograph of apparatuses of two sizes according to embodiments of the present disclosure. The apparatuses of FIG. 5b were used in the examples below.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition or the limitation of the invention.

FIG. 1 shows a reaction scheme of the conversion pathway from HMF to FDCA in the water phase.

FIG. 2 shows an illustration of a triphasic system for the direct conversion of carbohydrates such as sugars to furandicarboxylic acid such as FDCA.

FIG. 3 shows schematic illustrations of examples of triphasic reactors.

FIG. 4 shows (a) a photograph of the triphasic reaction setup in Example 2a, (b) the obtained FDCA yield vs. reaction time, (c) the HPLC detection results for the reaction in phase III after 5 h, 10 h, 20 h and 30 h, and (d) the HPLC detection results for the reaction in phase III after 5 h.

FIG. 5 shows in (a) a schematic design of an apparatus in accordance with an embodiment of the present disclosure, and (b) a photograph of the apparatuses used in the examples.

FIG. 6 shows the TEM and XRD of a prepared Au₈Pd₂/HT catalyst used in the examples.

FIG. 7 shows a ¹H NMR spectrum of isolated FDCA product prepared from Example 2a.

### EXAMPLES

Non-limiting examples of the invention and a comparative example will be further described in greater detail, which should not be construed as in any way limiting the scope of the invention.

Materials:

The carbohydrates used in the examples were D-Glucose and D-Fructose from Alfa Aesar (Massachusetts, USA). TEAB, HMF, FDCA, and Amberlyst®-15 were purchased from Sigma-Aldrich (Missouri, USA). MIBK was purchased from Merck (New Jersey, USA).

All the chemicals were used directly without any pre-treatment.

Product Analysis:

In the examples, HMF and FDCA were analyzed by HPLC (Agilent Technologies, California, USA, 1200 series) and confirmed with isolation yield. HPLC working conditions were column (Agilent Hi-Plex H, 7.7×300 mm, 8 μm), solvent 10 mM H₂SO₄, flow rate 0.7 ml/min, 25° C., UV detector, 280 nm for HMF and 254 nm for FDCA. The retention times for detected compounds were 20.7 min, 24.4 min, 29.4 min and 36.5 min for FDCA, HFCA, FFCA and HMF, respectively. Fructose and glucose were measured using a Sugar Analyzer (DKK-TOA Corporation, Japan. Model: SU-300).

Characterization:

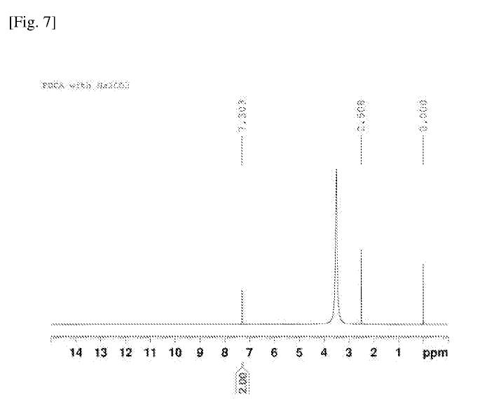

In the examples, the product was characterized by ¹H and ¹³C NMR (Bruker, Massachusetts, USA, AV-400). The Au₈Pd₂/HT catalyst was characterized by TEM (FEI Tecnai F20) and XRD (PANalytical x-ray diffractometer, X'pert PRO, with Cu Kα radiation at 1.5406 Angstroem). The TEM and XRD characterization results of the Au₈Pd₂/HT catalyst are shown in FIG. 6.

### Example 1

The Au₈Pd₂/HT catalyst was prepared in this example.

Au₈Pd₂/HT was prepared according to a known method (G. S. T. Yi, S. P.; Li, X. K.; Zhang, Y. G., ChemSusChem 2014).

0.1 mmol of HAuCl₄ and 0.025 mmol of NaPdCl₄ were dissolved in 40 ml of water. To this solution, 1 g of hydrotalcite was added, followed by addition of NH₃ aqueous solution (29.5%, 0.425 ml) until pH=10. The solution was vigorously stirred for 6 h and refluxed for 30 min at 373 K. The resulting solid was filtered, washed thoroughly with water and heated at 473 K overnight.

### Example 2a

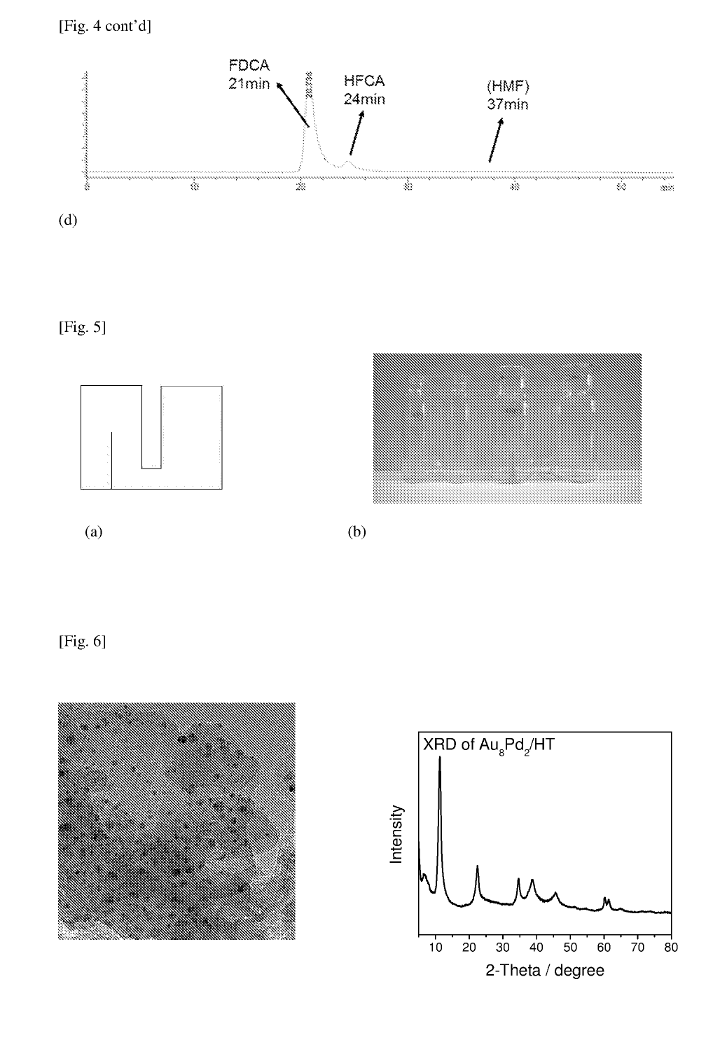

A one-pot conversion of fructose to FDCA in a triphasic reactor (shown in FIG. 5b) was conducted in this example. A photograph of the triphasic reaction setup with reactants used in this example is shown in FIG. 4a.

0.18 g fructose (1 mmol), 0.91 g TEAB, 0.09 ml water, and 0.018 g smashed amberlyst-15 were added to phase I of the reactor. The reactor was pre-heated to 95° C. and stirred with a magnetic stirrer to melt and mix all the reactants.

0.25 g Au₈Pd₂/HT catalyst, 0.106 g of Na₂CO₃ (1 mmol), and 10 ml of water were added to the other side of the reactor (phase III).

4 ml of MIBK was added on top of phase I and phase III.

The reactor was put in an oil bath pre-heated to 95° C.

Oxygen gas was bubbled into phase III during the reaction, with water added if the water level decreased. Every 5 hours, an aliquot of solution was taken out from phase III (right chamber shown in FIG. 4a) for HPLC analysis. The reaction was conducted for 30 hours. FIG. 4b shows the FDCA yield versus reaction time. For the first 10 hours, FDCA yield increased almost linearly over time. The FDCA yield topped at 20 hours with 78% FDCA overall yield. Thereafter, the FDCA yield decreased slowly at the 25-hour and 30-hour points, which may due to the degradation of FDCA over prolonged reaction time.

The FDCA product was isolated and analysed in Na₂CO₃ by ¹H NMR and the characterization results are shown in FIG. 7.

The reaction progress of the triphasic system was also monitored by analysis of the FDCA yield in phase III with HPLC. As shown in FIG. 4c, the FDCA yield gradually increased from 5 hours to 20 hours and reached a maximum yield of 78% at 20 hours.

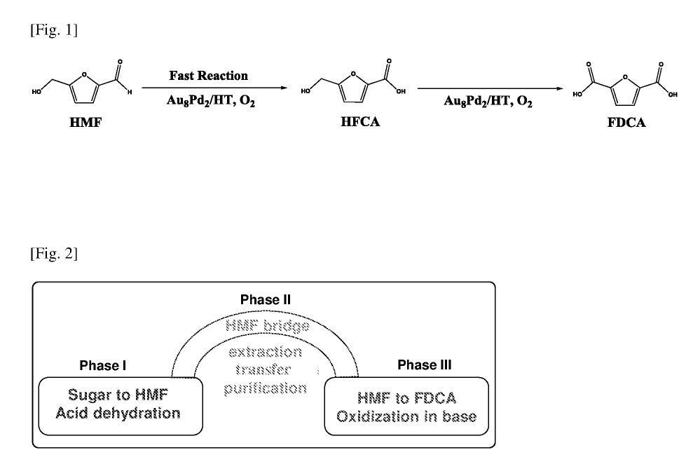

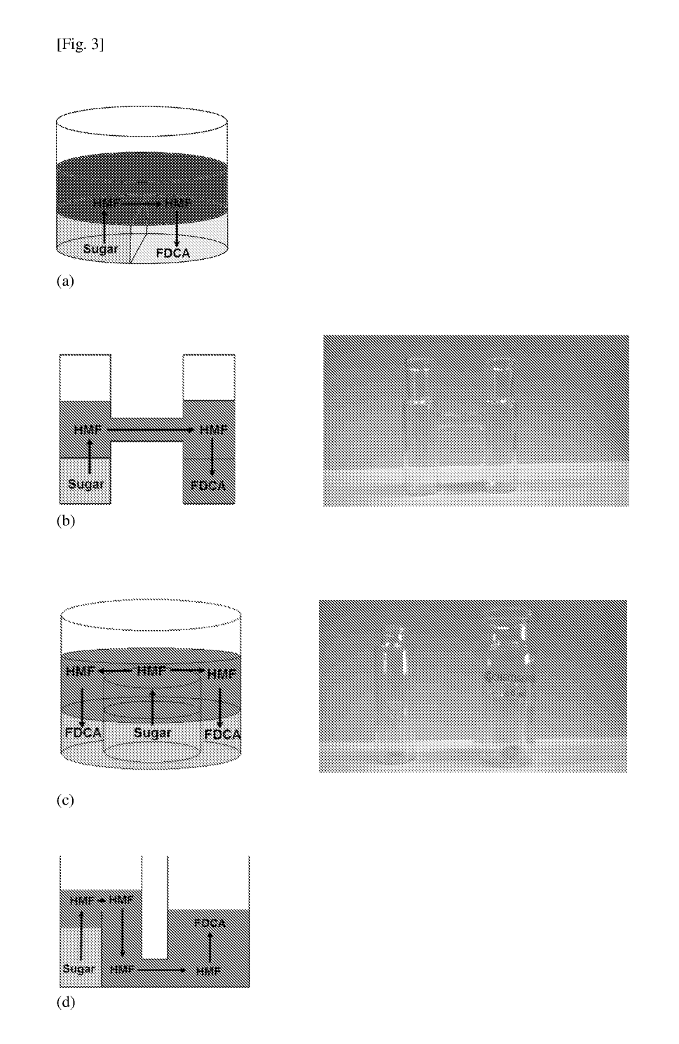

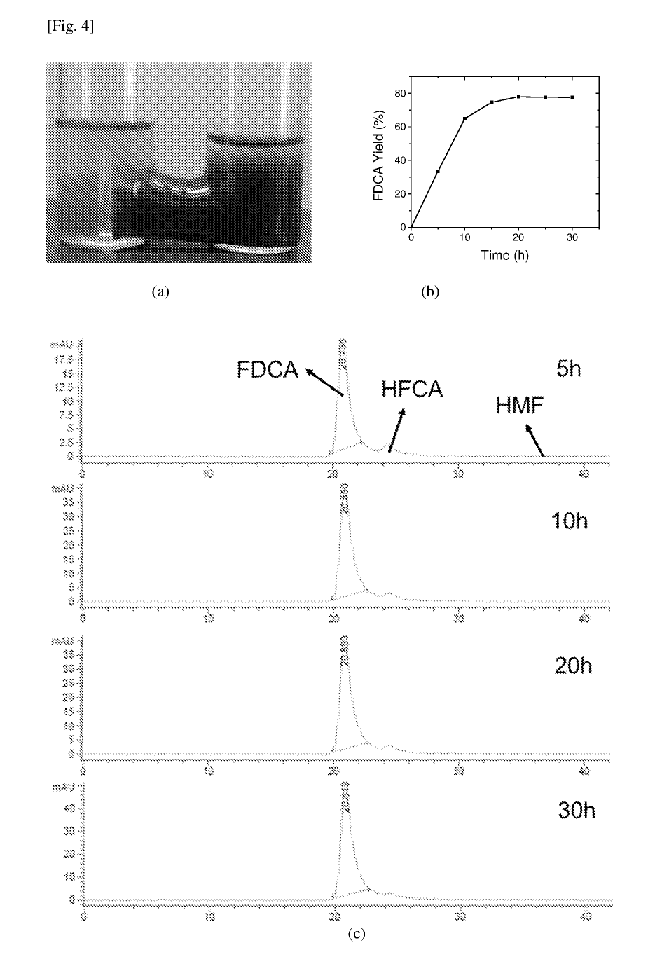

As shown in FIG. 4d, after 5 hours of reaction in phase III, only HFCA (retention time at 24 min) and FDCA (retention time at 21 min) could be detected. Almost no HMF was observed (HMF retention at 37 min). As expected, a high content of HMF was detected only in MIBK (phase II) and TEAB (phase I) (results not shown). This indicates that the conversion of HMF to FDCA is via the HFCA intermediate (as shown in FIG. 1), and the conversion from HMF to HFCA is fast. Once HMF was diffused to phase III, it was quickly converted to HFCA, and then converted to FDCA.

### Example 2b

To study the kinetic process in the triphasic reactor of Example 2a, a step-by-step reaction was conducted, using the same amounts of chemicals as in Example 2a.

Firstly, the conversion of fructose to HMF in TEAB was carried out according to a known method (S. P. Simeonov, J. A. S. Coelho, C. A. M. Afonso, ChemSusChem 2012, 5, 1388-1391) but modified by using a lower reaction temperature of 95° C. This is in the consideration of the reaction in phase III, where the optimized reaction temperature is 95° C.

The conversion of fructose to HMF in TEAB was a fast reaction. It was completed after 30 min with HMF yield of 86% in this example.

The reaction was then upgraded to a bi-phasic system, with 4 ml of MIBK added on top as an extraction layer. TEAB is immiscible with MIBK and thus, a clear interface between TEAB and MIBK was maintained during the reaction. After 30 min reaction at 95° C., for 1 mmol of fructose, 0.6 mmol of HMF was detected in TEAB, and 0.22 mmol of HMF in MIBK. The HMF distribution ratio between MIBK and TEAB was therefore about 1:2.7.

Separately, the conversion of HMF (prepared from fructose) to FDCA was conducted in 10 ml water, with 0.25 g of Au₈Pd₂/HT catalyst and 1 mmol Na₂CO₃. The reaction was conducted at 95° C. with O₂ bubbling and was completed in 7 hours with almost quantitative yield of FDCA.

As described above and in FIG. 4b, the whole process from fructose to FDCA was completed at nearly 20 hours, with a total yield of 78% FDCA. This indicates that the mass transfer of HMF from phase I to phase III via MIBK was the bottle neck, which slowed down the whole process.

### Example 3

In this example, the conversion of glucose to FDCA was performed. The direct conversion of glucose to FDCA in a triphasic reactor is more challenging than the conversion of fructose to FDCA, as glucose needs to be isomerized to fructose.

In the triphasic reactor (shown in FIG. 5b), 0.18 g glucose (1 mmol), 0.91 g TEAB, 0.09 ml water, 0.018 g smashed amberlyst-15 and 0.0266 g CrCl₃.6H₂O (0.1 mmol) were added to phase I of the reactor to convert glucose to HMF. TEAB was used as the reaction media and amberlyst-15/CrCl₃ was selected as catalysts.

Phase I was initially conducted at 95° C. However, after 7 hours of reaction, only negligible amount of FDCA was detected, with the glucose conversion at only 7.2%. The low glucose conversion may due to the low reaction temperature in phase I.

The reaction in phase I was improved upon by conducting the reaction at 120° C. for 30 min. To achieve this, the triphasic reactor setup was tilted to heat only the phase I chamber of the reactor. After that, the temperature was lowered down to 95° C., and the whole reactor was heated in the same oil bath.

0.25 g Au₈Pd₂/HT catalyst, 0.106 g of Na₂CO₃ (1 mmol), and 10 ml of water were used in the other side of reactor (phase III).

4 ml of MIBK was added on top of phase I and phase III.

Oxygen was bubbled in reactor III during the reaction, with water added if the water level decreased.

In this example, 50.2% of FDCA yield was achieved with a full conversion of glucose. The results are shown in Table 1 below.

TABLE 1

| Entry | Reactiontime (h) | HFCA yield (%) | FDCA yield (%) |
|---|---|---|---|
| 1 | 10 | 28.2 | 26.1 |
| 2 | 20 | 17.0 | 42.9 |
| 3 | 30 | 6.6 | 50.2 |
| 4 | 40 | 3.4 | 49.6 |
| 5 | 50 | 0.9 | 48.4 |

### Example 4

In this example, in the conversion of fructose to FDCA, saturated NaCl aqueous solution was used as the reaction media in phase I of the triphasic system.

The reaction conditions used were 0.18 g fructose, 0.6 ml 0.25 M HCl (NaCl saturated), 4 ml MIBK, 0.1 g Au—Pd/HT, 10 ml H₂O and 1 mmol Na₂CO₃.

The reaction was conducted at 95° C. and an overall FDCA yield of 41% was achieved, as shown below in Table 2.

TABLE 2

| Entry | Reaction time (h) | HFCA yield (%) | FDCA yield (%) |
|---|---|---|---|
| 1 | 5 | 13.3 | 12 |
| 2 | 10 | 14.8 | 26 |
| 3 | 20 | 2.9 | 41 |
| 4 | 30 | 0.9 | 38 |

In conclusion, a triphasic reactor that can convert sugars to FDCA in one-pot has been demonstrated. Overall FDCA yields of 78% and 50% were achieved with fructose and glucose feedstock, respectively. Kinetic studies showed that the phase transfer of HMF from phase I to phase III was the main bottle neck which slowed down the overall reaction.

### INDUSTRIAL APPLICABILITY

The one pot method of the invention may be useful as a method to convert sugars to furandicarboxylic acid. The high yield obtained in a simplified set-up may have a use for commercial production of furandicarboxylic acids derived from biomass. 2,5-furandicarboxylic acid can be made which has numerous applications as mentioned in the background section. An improved new apparatus has been further disclosed which allows for running the one pot process with good phase separation.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A one-pot method of producing furandicarboxylic acid from carbohydrate, the method comprising:
a) reacting the carbohydrate via a dehydration reaction to produce an intermediate in a first solvent phase;
b) contacting the first solvent phase with a second solvent phase at a first contact area;
c) extracting the intermediate to the second solvent phase;
d) contacting the second solvent phase directly with a third solvent phase at a different contact area;
c) oxidizing the intermediate to produce the furandicarboxylic acid in the third solvent phase.

2-39. (canceled)

40. The method of claim 1, wherein the carbohydrate is selected from the group consisting of glucose, fructose and cellulose; preferably glucose or fructose; or more preferably,
the intermediate is 5-hydroxymethylfurfural; or more preferably,
the furandicarboxylic acid is 2,5-furandicarboxylic acid.

41. The method of claim 1, wherein the first solvent phase is tetraethylammonium bromide.

42. The method of claim 1, wherein the first solvent phase is an aqueous solution, preferably comprising NaCl.

43. The method of claim 1, wherein the second solvent phase is selected to allow diffusion of the intermediate through the second solvent phase to the third solvent phase.

44. The method of claim 1, wherein the second solvent phase is selected to at least partially chemically isolate the dehydration step and the oxidation step and is optionally selected to be immiscible with the first solvent phase and the third solvent phase.

45. The method of claim 1, wherein the second solvent phase is capable of dissolving the intermediate.

46. The method of claim 1, wherein the second solvent phase is selected to reduce prevent furandicarboxylic acid from dissolving therein.

47. The method of claim 1, wherein the second solvent phase is an organic solvent, preferably being selected from C₄₋₆ alkyl alcohol, C₃₋₈ alkyl ketone and mixtures thereof and most preferably is methyl isobutyl ketone or ethyl methyl ketone.

48. The method of claim 1, wherein the distribution ratio of 5-hydroxymethylfurfural in the first solvent phase and the second solvent phase is more than about 0.1, and preferably about 1.5 to about 3.5.

49. The method of claim 1, wherein the third solvent phase is capable of dissolving furandicarboxylic acid.

50. The method of claim 1, wherein the third solvent phase is an aqueous solution which optionally comprises sodium carbonate.

51. The method of claim 1, wherein the oxidation step is carried out in the presence of oxygen and a catalytic system, wherein the catalytic system is preferably a supported catalytic system comprising gold-palladium/hydrotalcite and more preferably Au₈Pd₂/hydrotalcite.

52. The method of claim 1, wherein the oxidation step is conducted at a temperature of about 95° C. and optionally comprises converting the intermediate in the third solvent phase to a second intermediate, preferably 5-hydroxymethyl-2-furancarboxylic acid, which is optionally converted to furandicarboxylic acid in the third solvent phase.

53. The method of claim 1, wherein the carbohydrate is glucose and wherein the dehydration step is carried out in the presence of a catalytic system comprising an acidic ion exchange resin and CrCl₃ and is optionally conducted at a temperature of about 90° C. to about 100° C.; or is further optionally conducted at a temperature of about 110° C. to about 130° C.

54. The method of claim 1, wherein the carbohydrate is fructose and wherein the dehydration step is carried out in the presence of a catalytic system comprising an acidic ion exchange resin.

55. An apparatus for use in converting carbohydrate into furandicarboxylic acid in a one-pot process, the apparatus comprising:
a first chamber, which is preferably cylindrical in shape, fluidly connected to a second chamber, which is preferably cylindrical in shape and preferably of the same dimension as the first chamber, by a conduit,
wherein the first chamber comprises a dividing means, which preferably has a height of about 10% to about 50% of the height of the first chamber, to at least partially separate the first chamber into a first subzone and a second subzone,
wherein the first subzone defines a first reaction zone for producing an intermediate from the carbohydrate and the second chamber defines a second reaction zone for producing furandicarboxylic acid from the intermediate,
wherein the conduit, the dividing means and the second subzone are configured to at least partially chemically isolate the first and second reaction zones and wherein optionally the dividing means extends from the base of the first chamber.
